(19) United States
(12) Patent Application Publication
(10) Pub. No.: US 2025/0256038 A1
(43) Pub. Date: Aug. 14, 2025

(54) LIGHT-BASED VISUAL CUES THAT ASSIST IN MEDICATION DELIVERY TO A PATIENT

(71) Applicant: Janssen Research & Development, LLC, Raritan, NJ (US)

(72) Inventor: Jason TANSKY, Raritan, NJ (US)

(21) Appl. No.: 18/706,593

(22) PCT Filed: Oct. 17, 2022
(86) PCT No.: PCT/IB2022/059962

§ 371 Date: May 1, 2024

Related U.S. Application Data
(60) Provisional application No. 63/275,840, filed on Nov. 4, 2021.

(51) Int. Cl. A61M 5/315

(52) U.S. Cl. CPC A61M 5/3157; A61M 2205/332; A61M 2205/583; A61M 2205/587

(57) ABSTRACT

One or more light sources may be integrated with a medication delivery device, and states of the light sources may be changed. For example, a first lighting state change may be performed, such as based on a detection that an applied force meets or exceeds a threshold break force or an interaction of a movable component with a proximal sensor integrated with the medication delivery device. The first lighting state change may provide visual feedback indicating that an injection has begun. Additionally, a second lighting state change may be performed, such as based on a detection that a resistive force meets or exceeds a threshold back force or an interaction of the movable component with a distal sensor integrated with the medication delivery device. The second lighting state change may indicate that a complete dosage of the medication has been delivered from the medication delivery device.

### TECHNICAL FIELD

The present disclosure relates generally to providing light-based visual cues, for example to assist a patient in proper use of a medication delivery device, such as a medication injection device. Light sources, such as one or more organic light-emitting diodes (OLED's), may be activated, for example based on output from one or more sensors.

### BACKGROUND

A medication delivery device may be used to store and/or deliver a medication. In some examples, a medication delivery device may be a medication injection device, such as a prefilled syringe, an autoinjector, or wearable infusion pump. In some other examples, a medication delivery device may be a medication storage container, such as a bottle, a blister pack or other medication packaging.

Patients may often not perform an injection properly, such as by failing to remove a cap of the medication delivery device, not sufficiently pressing down on a medication delivery device to inject a complete dosage, failing to lift the medication delivery device up after injection, and the like. Additionally, patients may sometimes neglect to perform other medication delivery steps, such as shaking a medication before use.

Medication delivery instructions may sometimes be printed on a product label or instruction sheet. However, users may often neglect to read the printed instructions or may not completely comprehend the instructions. Moreover, it may be unclear from printed instructions exactly which step of a process the user is currently on, thereby potentially resulting in missed, or otherwise improperly performed, steps and instructions.

### SUMMARY

An example light-based medication delivery cueing system is described. The example system may comprise one or more force sensors. The one or more force sensors may measure an applied force that is applied, in an insertion direction, to a movable component of a medication delivery device, wherein the movable component is moved, in the insertion direction, to result in an injection of a medication into a patient. The one or more force sensors may further measure a resistive force that resists movement, in the insertion direction, of the movable component. The example system may further comprise one or more processing components. The one or more processing components may determine when the applied force becomes greater than, or equal to, a threshold break force that is required to initiate movement of the movable component in the insertion direction. The one or more processing components may further determine when the resistive force becomes greater than, or equal to, a threshold back force that stops the movement of the movable component in the insertion direction. The example system may further comprise a first light source. A first lighting state change of the first light source may be performed based on the applied force becoming greater than, or equal to, the threshold break force. A second lighting state change may be performed based on the resistive force becoming greater than, or equal to, the threshold back force

An example light-based medication delivery cueing method is described that may comprise measuring, by one or more force sensors, an applied force that is applied, in an insertion direction, to a movable component of a medication delivery device, wherein the movable component is moved, in the insertion direction, to result in an injection of a medication into a patient. The example method may further comprise measuring, by the one or more force sensors, a resistive force that resists movement, in the insertion direction, of the movable component. The example method may further comprise determining when the applied force becomes greater than, or equal to, a threshold break force that is required to initiate movement of the movable component in the insertion direction. The example method may further comprise performing a first lighting state change of a first light source based on the applied force becoming greater than, or equal to, the threshold break force. The example method may further comprise determining when the resistive force becomes greater than, or equal to, a threshold back force that stops the movement of the movable component in the insertion direction. The example method may further comprise performing a second lighting state change based on the resistive force becoming greater than, or equal to, the threshold back force.

Another example light-based medication delivery cueing system is described. The example system may comprise one or more proximal sensors that detect a first interaction of a movable component of a medication delivery device with the one or more proximal sensors. The movable component may be moved, in an insertion direction, to result in an injection of a medication into a patient. The example system may further comprise one or more distal sensors that detect a second interaction of the movable component with the one or more distal sensors. When being moved in the insertion direction to result in the injection, the movable component may interact with the one or more distal sensors after interacting with the one or more proximal sensors. The example system may further comprise a first light source. A first lighting state change of the first light source may be performed based on the first interaction, and a second lighting state change may be performed based on the second interaction.

Another example light-based medication delivery cueing method is described that may comprise detecting, by one or more proximal sensors, a first interaction of a movable component of a medication delivery device with the one or more proximal sensors. The movable component may be moved, in an insertion direction, to result in an injection of a medication into a patient. The example method may further comprise performing, based on the first interaction, a first lighting state change to a first light source. The example method may further comprise detecting, by one or more distal sensors, a second interaction of the movable component with the one or more distal sensors. When being moved in the insertion direction to result in the injection, the movable component may interact with the one or more distal sensors after interacting with the one or more proximal sensors. The example method may further comprise performing, based on the second interaction, a second lighting state change.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1A is a front elevation view of a first example medication delivery device in a pre-use position.

FIG. 1B is a front elevation view of the first example medication delivery device with the cap removed so as expose the needle guard.

FIG. 1C is a front elevation view of the first example medication delivery device with the needle guard moved from its position in FIG. 1B.

FIG. 1D is a front elevation view of the first example medication delivery device with the upper housing moving toward the dispensed position.

FIG. 1E is a front elevation view of the first example medication delivery device with the upper housing in the dispensed position.

FIG. 1F is a front elevation view of the first example medication delivery device with the needle guard in the final position.

FIG. 2 is a view of a second example medication delivery device that is an example pre-filled syringe.

FIG. 3 is a view of a third example medication delivery device that is an example autoinjector.

FIG. 4 is a view of a fourth example medication delivery device that is an example wearable medication delivery device.

FIG. 5 is a view of example light sources and an example force sensor integrated with the first example medication delivery device.

FIG. 6 is a diagram of a first example light-based medication delivery cueing system.

FIG. 7 is flowchart showing a first example light-based medication delivery cueing process.

FIG. 8 is a view of an example light source and an example proximal sensor and example distal sensor integrated with the first example medication delivery device.

FIG. 9 is a diagram of a second example light-based medication delivery cueing system.

FIG. 10 is flowchart showing a second example light-based medication delivery cueing process.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims. Further, the term “at least one” stated structure as used herein can refer to either or both of a single one of the stated structure and a plurality of the stated structure. Additionally, reference herein to a singular “a,” “an,” or “the” applies with equal force and effect to a plurality unless otherwise indicated. Similarly, reference to a plurality herein applies with equal force and effect to the singular “a,” “an,” or “the.”

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

### Example Medication Delivery Devices

Some example medication delivery devices will now be described in association with which the light-based visual cueing techniques described may be implemented. The term medication delivery device, as used herein, refers to a device that is used to store and/or deliver a medication. Medication delivery devices may include, for example, medication injection devices and medication storage containers. In some examples, the light-based visual cueing techniques described herein may be implemented in association with medication injection devices such as autoinjectors, prefilled syringes, wearable medication delivery devices (e.g., patch pumps, on body delivery systems (OBDS's), etc.), and others. It is noted, however, that the techniques described herein are not limited to use with any particular types of medication delivery devices, and a wide variety of types of medication delivery devices may be employed.

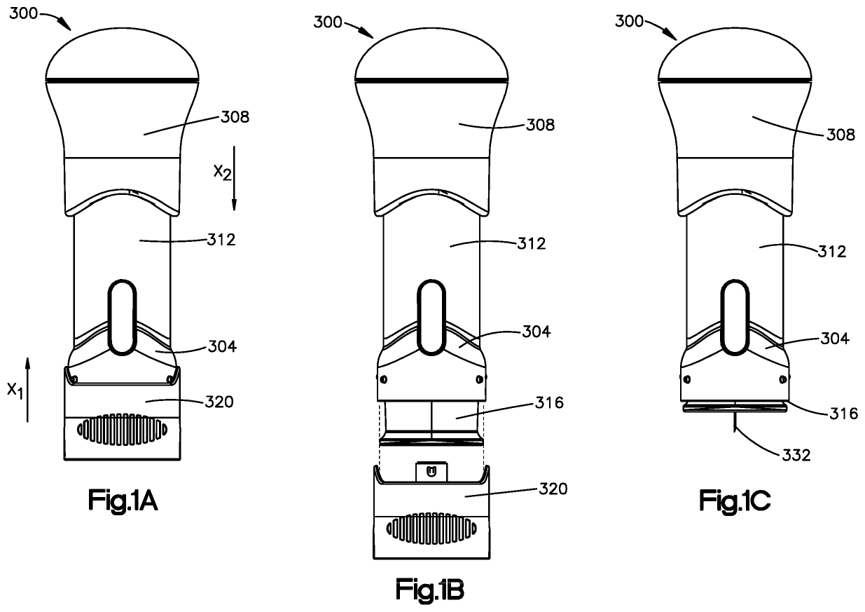

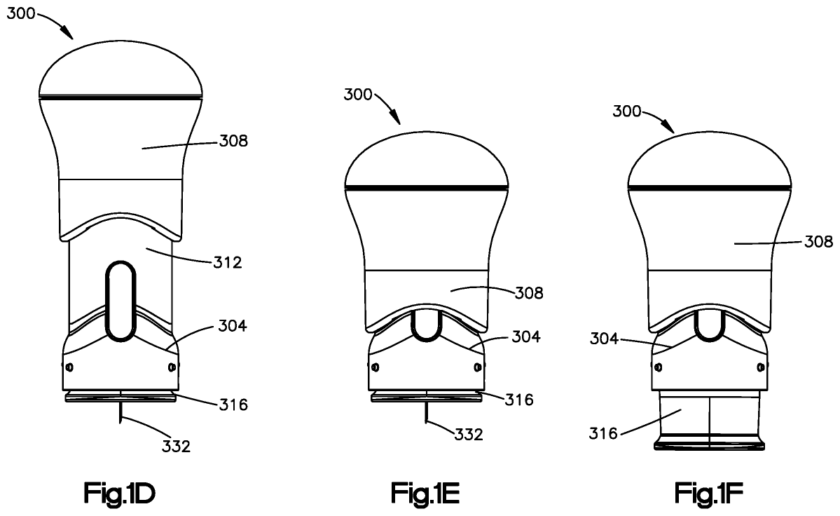

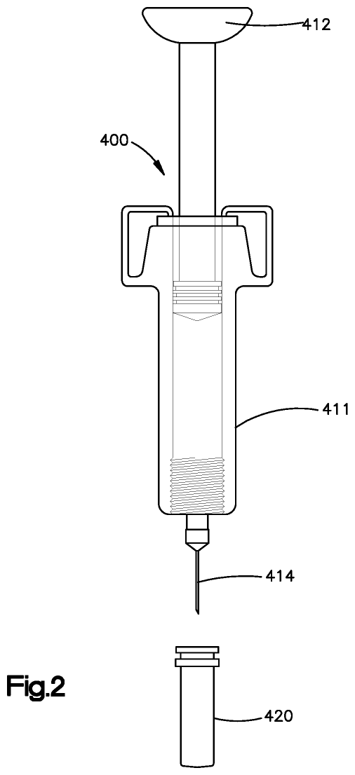

Referring now to FIGS. 1A-1F, a first example medication delivery device is described in association with which the light-based visual cueing techniques described may be implemented. As will be described, the device 300 may be employed to inject a medication, such as by pressing down on an upper housing 308 and moving the upper housing from a pre-use position to a dispensed position. In operation and in reference to FIGS. 1A-1F, the device 300 can be configured to deliver a medication. Prior to use, the upper housing 308 can be locked in the pre-use position, and the cap 320 can be coupled to the lower housing 304 so as to shield the needle guard 316 and the needle 332. When the device 300 is ready to be used, the cap 320 can be removed from the lower housing 304 as shown in FIG. 1B.

As shown in FIG. 1C, the device 300 can be positioned against a skin surface and a manual force can be applied to the upper housing 308 along an insertion direction, which is direction X₂ in FIG. 1A, such that, as the needle guard 316 is pressed against the skin surface, the needle guard 316 moves and the needle 332 is inserted into the tissue. As the needle guard 316 moves, the upper housing 308 may be unlocked from the pre-use position. As shown in FIGS. 1D and 1E, the upper housing 308 can then be moved along the insertion direction and over the middle housing 312. The upper housing 308 of device 300 is supported relative to the lower housing 304 and is configured to receive a manual force and move with respect to the lower housing 304 in the insertion direction from the pre-use position to the dispensed position in response to the manual force. The device 300 may include an internal syringe that is supported by the lower housing 304 and a plunger rod that is carried by the upper housing 308 and movable with the upper housing 308 so as to advance relative to the syringe when the upper housing 308 is moved along the insertion direction. The syringe may retain a medication and carry the needle 332 that is configured to be inserted into tissue. Advancement of the plunger rod relative to the syringe may cause the syringe to deliver the medication out the needle 332 and into the tissue. When the upper housing 308 reaches the dispensed position, the upper housing 308 may be locked in the dispensed position, for example via internal locking latches and latch members, so as to prevent re-use of the device 300. As the upper housing 308 is locked in dispensed position, such as by locking latches snapping over the latch members, an audible click may be produced that signifies to the user that the upper housing 308 has reached the dispensed position and is locked in the dispensed position. The upper housing 308 can be permanently locked in the dispensed position such that the device 300 is not reusable. It should be appreciated, however, that the upper housing 308 can be temporarily locked such that the device 300 can be sterilized and reused.

As shown in FIG. 1F, when the device 300 is removed from the skin surface along a direction X₁ opposite the insertion direction the needle guard 316 moves along the insertion direction to the final position. When in the final position, the needle guard 316 can be permanently locked in the final position so that the device 300 is not reusable. It should be appreciated, however, that the needle guard 316 can be temporarily locked such that the device 300 can be sterilized and reused.

Thus, device 300, which is described above with reference to FIGS. 1A-1F, is one example of a medication delivery device in association with which the light-based visual cueing techniques described herein may be implemented. However, the light-based visual cueing techniques described herein may also be implemented in association with other medication delivery devices, such as autoinjectors, prefilled syringes, wearable medication delivery devices (e.g., patch pumps, OBDS's), and others.

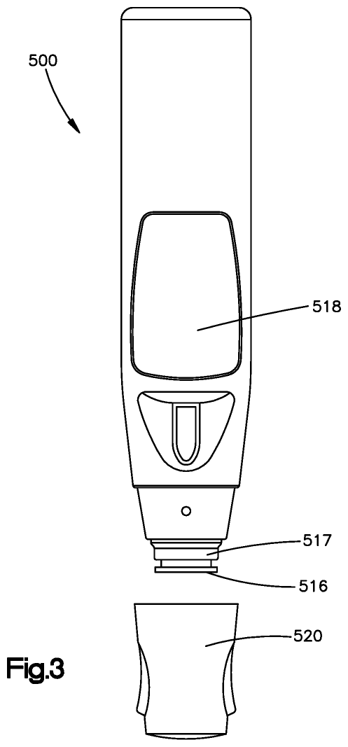

Referring now to FIG. 2, device 400 is an example of a prefilled syringe in association with which the light-based visual cueing techniques described herein may be implemented. As shown in FIG. 2, device 400 has a cap 420, which may be removed by the user when the user is ready to inject a medication using the device 400. After removing the cap 420, the device 400 may be held, and the needle 414 may be inserted. The user may then place his or her thumb on the plunger 412 and press the plunger 412 all the way down until the plunger 412 stops. The user may then release pressure from the plunger 412. The needle 414 may then retract into the body 411. Thus, in device 400, the user presses down on the plunger 412, for example as opposed to upper housing 308 of device 300 of FIG. 3.

Referring now to FIG. 3, device 500 is an example of an autoinjector in association with which the light-based visual cueing techniques described herein may be implemented. As shown in FIG. 2, device 500 has a cap 520, which may be removed by the user when the user is ready to inject a medication using the device 500. After removing the cap 520, the user may hold device 500 and position the device 500 against the skin with the safety sleeve 516 flat against the skin. The user may then push firmly against the skin, which may cause the safety sleeve 516 to slide into the cover 517. The user may then press button 518, which may cause a medication to be injected into the user via a hidden needle, such as may be located adjacent to safety sleeve 516 and cover 517. The device 500 may produce an audible sound, such as a first click sound, as the button 518 is pressed and the injection of the medication is started. The user may continue to press button 518 until the medication is fully dispensed. Similar to device 300 of FIG. 3, device 500 may also produce an audible sound, such as a click sound, once the medication has been fully dispensed, and this second click sound may be an indication to the user that the medication has been fully dispensed. Upon hearing the second click sound, the user may lift the device 500 from the skin. Thus, one way in which device 500 differs from device 300 is that device 500 allows the injection to be triggered by pressing a button 518.

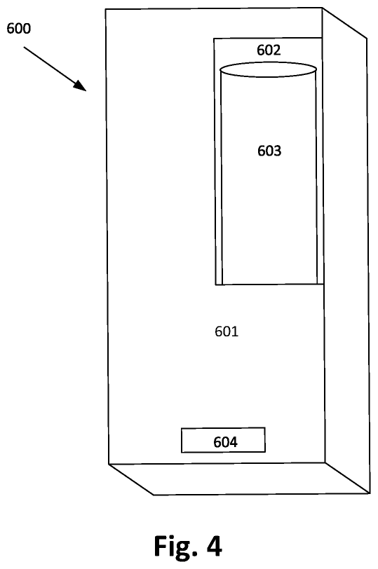

Referring now to FIG. 4, device 600 is an example of a wearable medication delivery device in association with which the light-based visual cueing techniques described herein may be implemented. Device 600 may be, or may be included in, an on body delivery system (OBDS). FIG. 4 shows a front surface 601 of device 600. In some examples, device 600 may be attachable to a user's skin. For example, in some cases, a rear surface of device 600, which may be opposite front surface 601 (and which is not shown in FIG. 4), may have an attached adhesive pad that may allow attachment of device 600 to a user's skin. Also, in some examples, device 600 may have an associated strap for assistance in attaching device 600 to a user's skin. As shown in FIG. 4, a medication tube 603 is visible through window 602. In operation, device 600 may cause a medication to be delivered from medication tube 603 to a user via an injection. In the example of FIG. 4, a user may press button 604 to cause the injection process to start. Device 600 may include a needle, such as may protrude from the rear of device 600, through which injection of the medication may be delivered.

Thus, as described above, the light-based visual cueing techniques described herein may be implemented in association with medication delivery devices such as autoinjectors, prefilled syringes, wearable medication delivery devices (e.g., patch pumps, on body delivery systems (OBDS's), etc.), and others. However, it is again noted that the techniques described herein are not limited to use with any particular types of medication delivery devices, and a wide variety of types of medication delivery devices may be employed. As another example, the light-based visual cueing techniques described herein may also be used in combination with other medication delivery devices, such as medication storage containers (e.g., storage bottles, blister packs and other storage packages, etc.).

### Light-Based Visual Cueing for Medication Delivery

As described herein, light sources may be integrated with a medication delivery device. In some examples, in order to not cause substantial changes to the size and/or shape of the medication delivery device, any or all of the light sources described herein may be a thin, flat and/or flexible component, such as an organic light-emitting diode (OLED) and/or OLED display. Also, in some examples, other light sources may be employed, such as a light-emitting diode (LED), an LED display, other thin film type lights, incandescent lights, and/or other light sources. As also described herein, a state of a light source may be changed based on one or more sensors, such as one or more force sensors. A force sensor is a sensor that senses force, such as by converting an input, load, weight, tension, compression and/or pressure into one or more electrical output signals. The state of the light source may be changed, such as by causing the light source to start or stop emitting light, causing the light source to start or stop flashing, causing the light source to change color, and in other ways. A lighting state change, which is a change in state of one or more light sources, may be performed in order to assist users with the injection process, such as by alerting users when an injection has begun, when an injection is continuing, and when an injection is completed. Patients and other users may often not perform an injection properly, such as by not sufficiently pressing down on a medication delivery device to inject a complete dosage. By using light-based alerting of the status of the injection process to users, the likelihood is reduced that users may perform the injection process improperly.

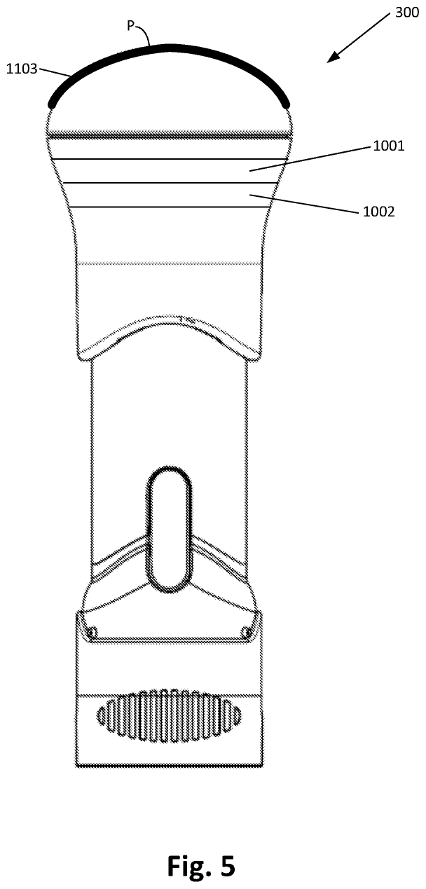

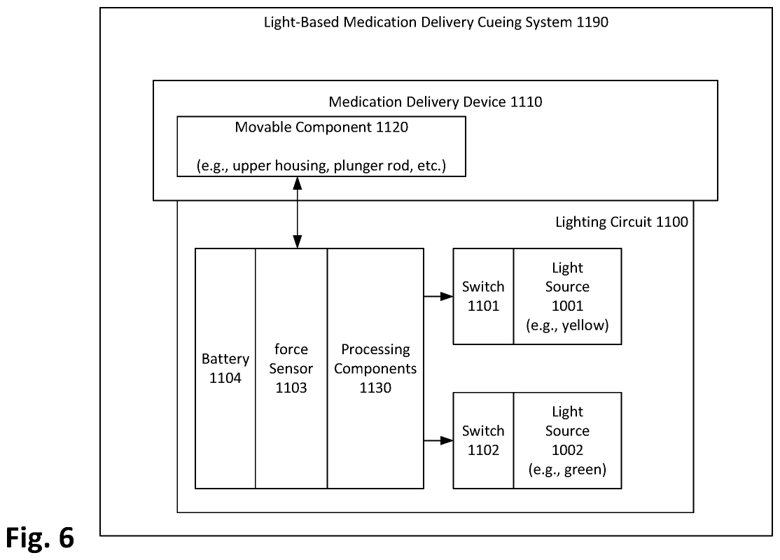

Referring now to FIG. 5, an example is shown of a light source 1001 and a light source 1002 integrated with device 300. In some examples, light source 1001 and light source 1002 may be OLED's, OLED displays, LED's, or another type of light source. In some examples, light source 1001 and light source 1002 may have different colors than one another. In one specific example, light source 1001 may have a yellow color, and light source 1002 may have a green color. The device 300 also includes a force sensor 1103. As will be described in detail below with respect to FIG. 6, the force sensor 1103 may measure forces associated with device 300, such as to result in state changes to light sources 1001 and 1002. The force sensor 1103 may be located at, or adjacent to, the proximal end P of device 300.

Referring now to FIG. 6, an example of a light-based medication delivery cueing system 1190 will now be described in detail. As shown, the light-based medication delivery cueing system 1190 includes a lighting circuit 1100 and a medication delivery device 1110, such as device 300 of FIG. 5.

The lighting circuit 1100 may be integrated with the medication delivery device 1110. The term integrated with, as used herein, means that the lighting circuit 1100 is included within the medication delivery device 1110 or is otherwise directly, or indirectly (e.g., via one or more connecting or attaching components), physically connected, or attached, to (e.g., via an adhesive, etc.) the medication delivery device 1110, before and/or at the time that the medication delivery device is used by a patient (and/or by another user on the patient's behalf). Thus, battery 1104, force sensor 1103 processing components 1130, switch 1101, light source 1001, switch 1102, and light source 1002 may be integrated with the medication delivery device 1110. In some examples, the lighting circuit 1100 may be included in one or more thin and flexible adhesive labels that are attachable to, and/or attached to, the medication delivery device 1110. Thus, in some examples, any, or all, of battery 1104, force sensor 1103 processing components 1130, switch 1101, light source 1001, switch 1102, and light source 1002 may be included in one or more thin and flexible adhesive label that are attachable to, and/or attached to, the medication delivery device 1110. Additionally, in some examples, the lighting circuit 1100 may be included within the medication delivery device 1110. For example, the lighting circuit 1100 may be embedded into one or more other components of the medication delivery device 1110, such as by being molded into plastic and/or other materials of which those components may be comprised. Thus, in some examples, any, or all, of battery 1104, force sensor 1103 processing components 1130, switch 1101, light source 1001, switch 1102, and light source 1002 may be included within the medication delivery device 1110.

The medication delivery device 1110 incudes a movable component 1120. For example, as described above with reference to FIGS. 1A-1F, the upper housing 308 of device 300 is supported relative to the lower housing 304 and is configured to receive a manual force and move with respect to the lower housing 304 in an insertion direction (e.g., direction X₂ in FIG. 1A) from a pre-use position to a dispensed position in response to the manual force. The device 300 may include an internal syringe that is supported by the lower housing 304 and a plunger rod that is carried by the upper housing 308 and movable with the upper housing 308 so as to advance relative to the syringe when the upper housing 308 is moved along the insertion direction. Thus, in some examples, the force sensor 1103 may detect the amount of force that is applied to the upper housing 308. As shown in FIG. 5, in order to assist in detecting the amount of force that is applied to the upper housing 308, the force sensor 1103 may be located at, or adjacent to, the proximal end P of device 300.

Movable component 1120 is a component of medication delivery device 1110 that is moved, in an insertion direction (e.g., direction X₂ in FIG. 1A), to result in an injection of a medication into a patient. To result in the injection, the movable component 1120 may be moved from a pre-use position to a dispensed position. In the specific example described above, upper housing 308 is an example of movable component 1120. Device 300, and other medication delivery devices, may also include this and/or other movable components, such as a plunger rod and/or a portion thereof (e.g., a thumb rest or other portion of the plunger rod), a needle shield and/or a portion thereof (e.g., a skin contact surface or other portion of a needle shield), a stopper rod and/or a portion thereof, etc. In some examples, the movable component 1120 may be directly contacted by a user. In other examples, the movable component 1120 may not be directly contacted by the user. For example, force may be transmitted from a component directly contacted by the user to the movable component 1120.

The lighting circuit 1100 includes a force sensor 1103. The force sensor 1103 may be configured to measure (e.g., to measure over time) an applied force that is applied to the movable component 1120 in the insertion direction. For example, the force sensor 1103 may measure the applied force at regular intervals (e.g., several times per second) and provide these measurements to processing components 1130. Processing components 1130 may be components that are programmed, or otherwise instructed, to perform the operations that they execute as described herein, and that may be included in one or more integrated circuit (IC) chips and/or other IC components.

A threshold break force may be required to initiate the movement of the movable component 1120 in the insertion direction. It is noted that the threshold break force may be different for different types of medication delivery devices. In some examples, the processing components 1130 may be programmed to include an indication of the threshold break force that is required for the given medication delivery device 1110 in which the processing components 1130 are included.

In some examples, based on measurements of the applied force from the force sensor 1103, the processing components 1130 may determine when the applied force becomes greater than, or equal to, the threshold break force. When the applied force becomes greater than, or equal to, the threshold break force, this may be an indication that a user has begun the injection. In some examples, in response to determining that the applied force becomes greater than, or equal to, the threshold break force, the processing components 1130 may send a signal to cause a first lighting state change of light source 1001 to be performed, such as by causing the light source 1001 to start or stop emitting light, causing the light source 1001 to start or stop flashing, causing the light source 1001 to change color, and/or other state changes. This may provide visual feedback to the user indicating that the injection has begun.

In one specific example, the first lighting state change may include powering-on light source 1001, such as to cause light source 1001 to emit light. In FIG. 6, the force sensor 1103 and processing components 1130 are powered by battery 1104. The light source 1001 may initially be unpowered, and the light source 1001 may remain unpowered until the applied force becomes greater than, or equal to, the threshold break force. When the applied force becomes greater than, or equal to, the threshold break force, the light source 1001 may be powered-on. Switch 1101 is an electrical switch that may control the flow of power from the battery 1104 to the light source 1001. The switch 1101 may be initially in an open state (which does not allow power to be provided to the light source 1001) and then, upon determining that the applied force becomes greater than, or equal to, the threshold break force, moved to a closed state (which does allow power to be provided to the light source 1001). In some examples, the processing components 1130 may, upon determining that the applied force becomes greater than, or equal to, the threshold break force, provide a signal to close the switch 1101 and allow power to flow to the light source 1001. Processing components 1130 may additionally or alternatively provide one or more other signals, for example to light source 1001, to perform other state changes, such as color changes and the like.

In some examples, a threshold glide force may be required to continue the movement of the movable component 1120 in the insertion direction. For example, after movement of the movable component 1120 is initiated (e.g., via providing at least the threshold break force), at least the threshold glide force may be provided to cause further movement of the movable component 1120 in the insertion direction, thereby causing the injection process to continue. It is noted that the threshold glide force may be different for different types of medication delivery devices. In some examples, the processing components 1130 may be programmed to include an indication of the threshold glide force that is required for the given medication delivery device 1110 in which the processing components 1130 are included. In some examples, the threshold glide force may be greater than the threshold break force. In other examples, the threshold glide force may be equal to the threshold break force. In yet other examples, the threshold glide force may be less than the threshold break force.

In some examples, based on measurements of the applied force from the force sensor 1103, the processing components 1130 may determine when the applied force is greater than, or equal to, the threshold glide force. In some examples, the processing components 1130 may be programmed to not monitor for the threshold glide force until after it is determined that the threshold break force is met or exceeded. In some examples, in response to determining that the applied force is greater than, or equal to, the threshold glide force, a visual cue to continue the injection may be provided. In some examples, the visual cue is provided via light source 1001, such as via a state change of light source 1001. In one specific example, the visual cue includes a flashing of light source 1001, such as may be provided by repeatedly powering-off, and then powering-on, light source 1001. As described above, switch 1101 may be closed to cause power to be provided to light source 1001. Switch 1101 may also be opened to stop power from being provided to light source 1001. In some examples, the processing components 1130 may, upon determining that the applied force is greater than, or equal to, the threshold glide force, provide signals to repeatedly open and close the switch 1101 to cause flashing of the light source 1001.

The force sensor 1103 may also be configured to measure (e.g., to measure over time) a resistive force that resists movement, in the insertion direction, of the movable component 1120. For example, the force sensor may measure the resistive force at regular intervals (e.g., several times per second) and provide these measurements to processing components 1130.

A threshold back force is a force that stops the movement of the movable component 1120 in the insertion direction. The threshold back force may stop the movement of the movable component 1120 once the movable component 1120 is moved to a dispensed position. Thus, the threshold back force may correspond to a completion of the injection. Because it stops the movement of the movable component 1120, the threshold back force may be greater than the threshold glide force. It is noted that the threshold back force may be different for different types medication delivery devices. In some examples, the processing components 1130 may be programmed to include an indication of the threshold back force that is required for the given medication delivery device 1110 in which the processing components 1130 are included.

In some examples, based on measurements of the resistive force from the force sensor 1103, the processing components 1130 may determine when the resistive force becomes greater than, or equal to, the threshold back force. When the resistive force becomes greater than, or equal to, the threshold back force, this may be an indication that the complete dosage of the medication has been delivered from the medication delivery device 1110. In some examples, in response to the resistive force becoming greater than, or equal to, the threshold back force, a second lighting state change may be performed. The second lighting state change may be a visual cue to the user indicating that the complete dosage of the medication has been delivered from the medication delivery device 1110. The second lighting state change may include changing a state of light source 1001 and/or a state of light source 1002. In some examples, the second lighting state change may include ceasing the visual cue to continue the injection (e.g., the flashing of light source 1001), for example by powering off light source 1001 (or otherwise causing the light source 1001 to stop emitting light). Additionally or alternatively, in some examples, the second lighting state change may include changing a state of light source 1002.

In one specific example, the second lighting state change may include powering-on light source 1002, such as to cause light source 1002 to emit light. In FIG. 6, the light source 1002 may initially be unpowered, and the light source 1002 may remain unpowered until the resistive force becomes greater than, or equal to, the threshold back force. When the resistive force becomes greater than, or equal to, the threshold back force, the light source 1002 may be powered-on. Switch 1102 is an electrical switch that may control the flow of power from the battery 1104 to the light source 1002. The switch 1102 may be initially in an open state (which does not allow power to be provided to the light source 1002) and then, upon determining that the resistive force becomes greater than, or equal to, the threshold back force, moved to a closed state (which does allow power to be provided to the light source 1002). In some examples, the processing components 1130 may, upon determining that the resistive force becomes greater than, or equal to, the threshold back force, provide a signal to close the switch 1102 and allow power to flow to the light source 1002. Processing components 1130 may additionally or alternatively provide one or more other signals, for example to light source 1002, to perform other state changes, such as color changes and the like.

As described above, in some examples, light source 1001 and light source 1002 may have different colors than one another. In one specific example, light source 1001 may have a yellow color, and light source 1002 may have a green color. Thus, in these and other examples, causing light source 1002 to start emitting light may provide a clear visual cue to the user that the complete dosage of the medication has been delivered from the medication delivery device 1110. This may help to prevent the user from stopping the injection prematurely before the full dose is delivered. The causing light source 1002 to start emitting light may also cue to the user to stop pushing down on the medication delivery device 1110 and/or to lift up the medication delivery device 1110. In one specific example, the medication delivery device 1110 may include corresponding instructions, such as text that states, “stop pressing down when the lights on the side go from yellow to green.” In some examples, audio may be provided via an attached speaker, such as that indicates to stop pressing when the lights on the side go from yellow to green (or another similar indication). In an alternative embodiment, as opposed to both light sources 1001 and 1002, only light source 1001 be employed. For example, light source 1001 may be caused to stop emitting light (or otherwise have its state changed) based on the resistive force becoming greater than, or equal to, the threshold back force, and this may be a visual cue indicating that the complete dosage of the medication has been delivered from the medication delivery device 1110.

In some examples, any, or all, of the lighting circuit components (e.g., force sensor 1103, light source 1001, switch 1101, light source 1002, switch 1102, processing components 1130, battery 1104) of the lighting circuit 1100 (as well as other lighting circuits described herein) may be implemented, in whole or in part, using one or more integrated circuit (IC) components, such as one or more IC chips. The integrated circuit components may be connected to one or more circuit boards, such as a printed circuit board and/or flexible circuit board. The components may communicate and/or interact with one another via one or more electrical connections, for example via the one or more circuit boards. Any, or all, of the lighting circuit components of the lighting circuit 1100 shown in FIG. 6 (as well as other lighting circuits described herein) may optionally include one or more processing components (e.g., integrated with or separate from processing components 1130) and/or one or more memory components. The memory components can be volatile (such as some types of RAM), non-volatile (such as ROM, flash memory, etc.), or a combination thereof.

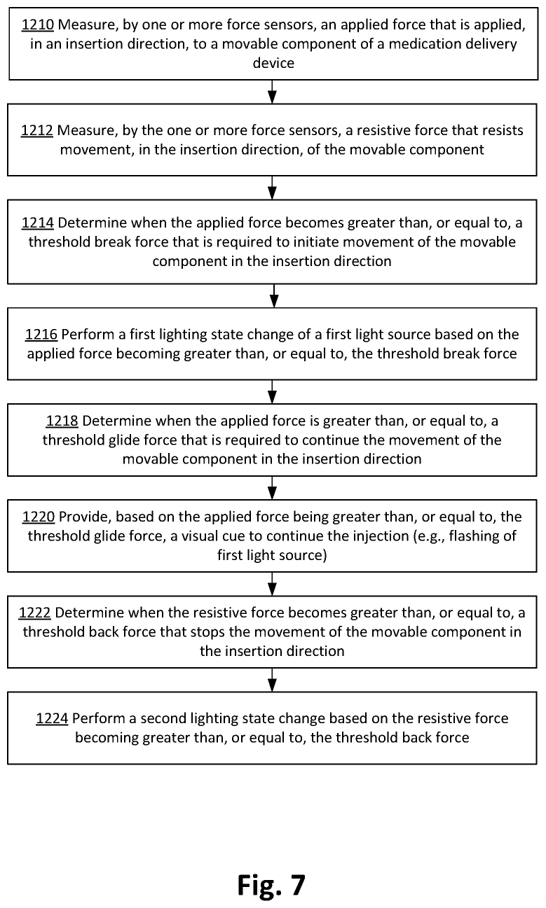

Referring now to FIG. 7, an example light-based medication delivery cueing process will now be described in detail. The process of FIG. 7 is initiated at operation 1210, at which an applied force that is applied, in an insertion direction, to a movable component of the medication delivery device is measured by one or more force sensors. The movable component is moved, in the insertion direction, to result in an injection of a medication into a patient. The applied force may be manually applied to a medication delivery device by a user. In the example of FIG. 6, the applied force is measured by force sensor 1103. For example, the force sensor 1103 may measure the applied force at regular intervals (e.g., several times per second) and provide these measurements to processing components 1130. At operation 1212, a resistive force that resists movement, in the insertion direction, of the movable component is measured by the one or more force sensors. In the example of FIG. 6, the resistive force is measured by force sensor 1103. For example, the force sensor 1103 may measure the resistive force at regular intervals (e.g., several times per second) and provide these measurements to processing components 1130. In some examples, at least one of the one or more force sensors may be contacted by a user during the injection. For example, a force sensor may be located on a surface of an upper housing of a medication delivery device that may be contacted by a user during the injection. Also, in some examples, at least one of the one or more force sensors may not be contacted by a user during the injection. For example, a force sensor may be internal to the medication delivery device. In some cases, a force sensor may be located between components of a medication delivery device (e.g., between upper housing 308 and a plunger rod, etc.), such as to measure force transmitted from one component contacted by the user to another component.

At operation 1214, it is determined when the applied force becomes greater than, or equal to, a threshold break force that is required to initiate movement of the movable component in the insertion direction. In the example of FIG. 6, based on measurements of the applied force from the force sensor 1103, the processing components 1130 may determine when the applied force becomes greater than, or equal to, the threshold break force. In some examples, the processing components 1130 may be programmed to include an indication of the threshold break force that is required for the given medication delivery device 1110 in which the processing components 1130 are included. When the applied force becomes greater than, or equal to, the threshold break force, this may be an indication that a user has begun the injection.

At operation 1216, a first lighting state change of the first light source is performed based on the applied force becoming greater than, or equal to, the threshold break force. The first light source may be, for example, an OLED, OLED display, LED, or another type of light source. In the example of FIG. 6, a first lighting state change of light source 1001 may be performed based on the applied force becoming greater than, or equal to, the threshold break force. In one specific example, the first lighting state change may include causing the first light source to start emitting light, such as by powering-on the first light source. Specifically, processing components 1130 may, upon determining that the applied force becomes greater than, or equal to, the threshold break force, send a signal to move switch 1101 from an open position to a closed position, such as to cause power to be provided to light source 1001. However, other state changes may also be performed. For example, the first lighting stage change may include causing the first light source to start emitting light, causing the first light source to stop emitting light, causing the first light source to start flashing, causing the first light source to stop flashing, causing the first light source to change color, and/or other state changes. The first lighting state change may provide visual feedback to the user indicating that the injection has begun.

At operation 1218, it is determined when the applied force is greater than, or equal to, a threshold glide force that is required to continue the movement of the movable component in the insertion direction. In the example of FIG. 6, based on measurements of the applied force from the force sensor 1103, the processing components 1130 may determine when the applied force is greater than, or equal to, the threshold glide force. In some examples, the processing components 1130 may be programmed to include an indication of the threshold glide force that is required for the given medication delivery device 1110 in which the processing components 1130 are included.

At operation 1220, a visual cue to continue the injection is provided based on the applied force being greater than, or equal to, the threshold glide force. In one specific example, the visual cue may include, for example, a flashing on and off of the first light source. In the example of FIG. 6, the visual cue may include flashing on and off of light source 1001. Specifically, processing components 1130 may, upon determining that the applied force is greater than, or equal to, a threshold glide force, send a signal to repeatedly move switch 1101 between the closed position and the open position, such as to cause the light source 1001 to flash on and off. In addition or as an alternative to flashing, the visual cue may include other state changes (e.g., color change, etc.).

At operation 1222, it is determined when the resistive force becomes greater than, or equal to, a threshold back force that stops the movement of the movable component in the insertion direction. In the example of FIG. 6, based on measurements of the resistive force from the force sensor 1103, the processing components 1130 may determine when the resistive force becomes greater than, or equal to, the threshold back force. In some examples, the processing components 1130 may be programmed to include an indication of the threshold back force that is required for the given medication delivery device 1110 in which the processing components 1130 are included. When the resistive force becomes greater than, or equal to, the threshold back force, this may be an indication that the complete dosage of the medication has been delivered from the medication delivery device 1110.

At operation 1224, a second lighting state change is performed based on the resistive force becoming greater than, or equal to, the threshold back force. The second lighting state change may be a visual cue to the user indicating that the complete dosage of the medication has been delivered from the medication delivery device. The second lighting state change may include changing a state of the first light source and/or a state of the second light source. The second light source may be, for example, an OLED, OLED display, LED, or another type of light source. In some examples, the second lighting state change may include ceasing the visual cue to continue the injection (e.g., the flashing of the first light source), for example by powering off the first light source (or otherwise causing the first light source to stop emitting light). Additionally or alternatively, in some examples, the second lighting state change may include changing a state of the second light source, such as by causing the second light source to start emitting light. In the example of FIG. 6, processing components 1130 may, upon determining that the resistive force has become greater than, or equal to, the threshold back force, send a signal to move switch 1102 from an open position to a closed position, such as to cause power to be provided to light source 1002. It is noted, however, that the second lighting state change may also include other state changes. For example, the second lighting stage change may include causing the first light source to start emitting light, causing the first light source to stop emitting light, causing the first light source to start flashing, causing the first light source to stop flashing, causing the first light source to change color, and/or other state changes. The second lighting stage change may also include causing the second light source to start emitting light, causing the second light source to stop emitting light, causing the second light source to start flashing, causing the second light source to stop flashing, causing the second light source to change color, and/or other state changes. The first light source may have a different color than the second light source. In one specific example, the first light source may have a yellow color, and the second light source may have a green color.

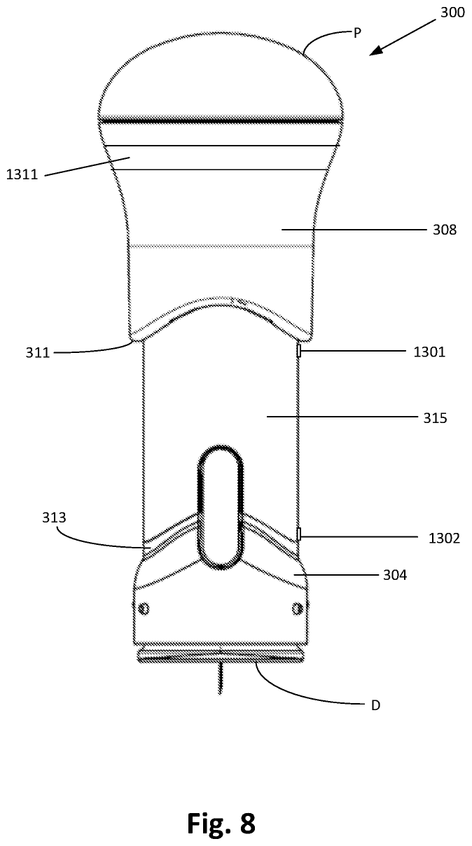

Referring now to FIG. 8, an example is shown in which a light source 1311, a proximal sensor 1301, and a distal sensor 1302 are integrated with device 300. Light source 1311 may be, for example, an OLED, OLED display, LED, or another type of light source. The proximal sensor 1301 and the distal sensor 1302 may be located on the middle housing body 315 of device 300. As shown, the proximal sensor 1301 is closer to the proximal end P of device 300 than is the distal sensor 1302. By contrast, the distal sensor 1302 is closer to the distal end D of device 300 than is the proximal sensor 1301. As the upper housing 308 moves from the pre-use position to the dispensed position to inject the medication, the upper housing 308 will interact first with the proximal sensor 1301 and then will subsequently interact with the distal sensor 1302. In some examples, the proximal senor 1301 and distal sensor 1302 may be sufficiently thin such that they do not block the movement path of the upper housing 308, though the upper housing 308 may contact the proximal senor 1301 and distal sensor 1302 as the upper housing is moved from the pre-use position to the dispensed position.

An interaction of the upper housing 308 (or other movable component) with the proximal sensor 1301, as that term is used herein, means that the upper housing 308 (or other movable component) is moved such that its movement is detected by the proximal sensor 1301. This may occur, for example, when the upper housing 308 (or other movable component) is moved along the middle housing body 315 such as to contact and/or cover the proximal sensor 1301. For example, in some cases, the proximal sensor 1301 may be a tactile and/or force sensor, and an interaction of the upper housing 308 (or other movable component) with the proximal sensor 1301 may be detected by the proximal sensor 1301 when the upper housing 308 (or other movable component) is moved such that it contacts the proximal sensor 1301. As another example, the proximal sensor 1301 may be a light sensor. In this example, an interaction of the upper housing 308 (or other movable component) with the proximal sensor 1301 may be detected by the proximal sensor 1301 when the upper housing 308 (or other movable component) is moved such that it fully or partially covers the proximal sensor 1301, thereby causing a reduction in light that is detected by the light sensor (as compared to the amount of light that was detected prior to the light sensor being covered by the upper housing 308). As another example, the proximal sensor 1301 may be a magnetic sensor. In this example, an interaction of the upper housing 308 (or other movable component) with the proximal sensor 1301 may be detected by the proximal sensor 1301 when the upper housing 308 (or other movable component) is moved such that a metallic component that may be included in the upper housing 308 (or other movable component) is moved within a threshold distance of the proximal sensor 1301. As should be appreciated, other types of sensors and corresponding interactions with a movable component may also be employed.

An interaction of the upper housing 308 (or other movable component) with the distal sensor 1302, as that term is used herein, means that the upper housing 308 (or other movable component) is moved such that its movement is detected by the distal sensor 1302. This may occur, for example, when the upper housing 308 (or other movable component) is moved along the middle housing body 315 such as to contact and/or cover the distal sensor 1301. For example, in some cases, the distal sensor 1302 may be a tactile and/or force sensor, and an interaction of the upper housing 308 (or other movable component) with the distal sensor 1302 may be detected by the distal sensor 1302 when the upper housing 308 (or other movable component) is moved such that it contacts the distal sensor 1302. As another example, the distal sensor 1302 may be a light sensor. In this example, an interaction of the upper housing 308 (or other movable component) with the distal sensor 1302 may be detected by the distal sensor 1302 when the upper housing 308 (or other movable component) is moved such that it fully or partially covers the distal sensor 1302, thereby causing a reduction in light that is detected by the light sensor (as compared to the amount of light that was detected prior to the light sensor being covered by the upper housing 308). As another example, the distal sensor 1302 may be a magnetic sensor. In this example, an interaction of the upper housing 308 (or other movable component) with the distal sensor 1302 may be detected by the distal sensor 1302 when the upper housing 308 (or other movable component) is moved such that a metallic component that may be included in the upper housing 308 (or other movable component) is moved within a threshold distance of the distal sensor 1302. As should be appreciated, other types of sensors and corresponding interactions with a movable component may also be employed.

While the example of FIG. 8 shows only a single proximal sensor 1301, there may, in some examples, be a plurality of proximal sensors. The plurality of proximal sensors may each be positioned at the same distance as one another from the distal end D, such that the upper housing simultaneously interacts with each of the plurality of proximal sensors. Additionally, while the example of FIG. 8 shows only a single distal sensor 1302, there may, in some examples, be a plurality of distal sensors. The plurality of distal sensors may each be positioned at the same distance as one another from the distal end D, such that the upper housing simultaneously interacts with each of the plurality of distal sensors.

As shown in FIG. 8, the proximal sensor 1301 may be positioned adjacent to the bottom edge 311 of the upper housing 308 when the upper housing 308 is in the pre-use position. This may allow the upper housing to interact with the proximal sensor 1301 immediately (or almost immediately) after the start of the injection of the medication. Similarly, the distal sensor 1302 may be positioned adjacent to the upper edge 313 of the lower housing 304. This may allow the upper housing to interact with the distal sensor 1302 immediately (or almost immediately) before fully injecting the dosage of the medication into the patient.

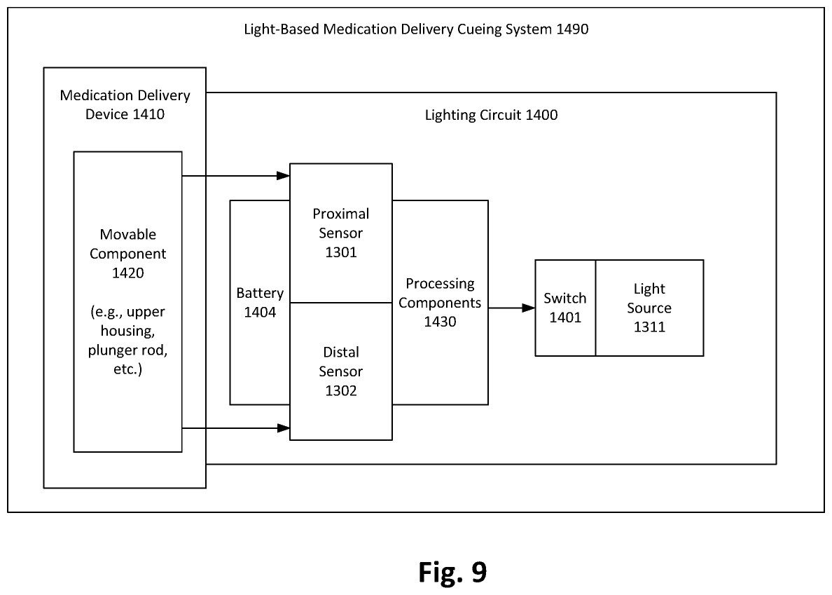

Referring now to FIG. 9, an example of a light-based medication delivery cueing system 1490 will now be described in detail. As shown, light-based medication delivery cueing system 1490 includes a lighting circuit 1400 and a medication delivery device 1410, such as device 300 of FIG. 8.

The lighting circuit 1400 may be integrated with the medication delivery device 1410. The term integrated with, as used herein, means that the lighting circuit 1400 is included within the medication delivery device 1410 or is otherwise directly, or indirectly (e.g., via one or more connecting or attaching components), physically connected, or attached, to (e.g., via an adhesive, etc.) the medication delivery device 1410, before and/or at the time that the medication delivery device is used by a patient (and/or by another user on the patient's behalf). Thus, battery 1404, proximal sensor 1301, distal sensor 1302, processing components 1430, switch 1401 and light source 1311 may be integrated with the medication delivery device 1410. In some examples, the lighting circuit 1400 may be included in one or more thin and flexible adhesive labels that are attachable to, and/or attached to, the medication delivery device 1410. Thus, in some examples, any, or all, of battery 1404, proximal sensor 1301, distal sensor 1302, processing components 1430, switch 1401 and light source 1311 may be included in one or more thin and flexible adhesive label that are attachable to, and/or attached to, the medication delivery device 1410. Additionally, in some examples, the lighting circuit 1400 may be included within the medication delivery device 1410. For example, the lighting circuit 1400 may be embedded into one or more other components of the medication delivery device 1410, such as by being molded into plastic and/or other materials of which those components may be comprised. Thus, in some examples, any, or all, of battery 1404, proximal sensor 1301, distal sensor 1302, processing components 1430, switch 1401 and light source 1311 may be included within the medication delivery device 1410.

The medication delivery device 1410 incudes a movable component 1420. For example, as described above with reference to FIGS. 1A-1F, the upper housing 308 of device 300 is supported relative to the lower housing 304 and is configured to receive a manual force and move with respect to the lower housing 304 in an insertion direction (e.g., direction X₂ in FIG. 1A) from a pre-use position to a dispensed position in response to the manual force. The device 300 may include an internal syringe that is supported by the lower housing 304 and a plunger rod that is carried by the upper housing 308 and movable with the upper housing 308 so as to advance relative to the syringe when the upper housing 308 is moved along the insertion direction.

Movable component 1420 is a component of medication delivery device 1410 that is moved, in an insertion direction (e.g., direction X₂ in FIG. 1A), to result in an injection of a medication into a patient. To result in the injection, the movable component 1420 may be moved from a pre-use position to a dispensed position. In the specific example described above, upper housing 308 is an example of movable component 1420. Device 300, and other medication delivery devices, may also include this and/or other movable components, such as a plunger rod and/or a portion thereof (e.g., a thumb rest or other portion of the plunger rod), a needle shield and/or a portion thereof (e.g., a skin contact surface or other portion of a needle shield), a stopper rod and/or a portion thereof, etc. In some examples, the movable component 1420 may be directly contacted by a user. In other examples, the movable component 1420 may not be directly contacted by the user. For example, force may be transmitted from a component directly contacted by the user to the movable component 1420.

The lighting circuit 1400 includes proximal sensor 1301, which detects when the movable component 1420 (e.g., upper housing 308 in the example of device 300) of the medication delivery device 1410 interacts with the proximal sensor 1301 as described above. The lighting circuit 1100 also includes distal sensor 1302, which detects when the movable component 1420 (e.g., upper housing 308 in the example of device 300) of the medication delivery device 1410 interacts with the distal sensor 1302 as described above.

In some examples, a first lighting state change of light source 1311 may be performed based on the proximal sensor 1301 detecting interaction of the movable component 1420 with the proximal sensor 1301. The first lighting state change of the light source 1311 may be a visual cue that the injection of the medication has begun. In one specific example, the first lighting state change may include powering-on the light source 1311, such as to cause the light source 1311 to start emitting light. However, other state changes may also be performed. For example, the first lighting stage change may include causing light source 1311 to start emitting light, causing light source 1311 to stop emitting light, causing light source 1311 to start flashing, causing light source 1311 to stop flashing, causing light source 1311 to change color, and/or other state changes. In some examples, upon detecting the interaction of the movable component 1420 with the proximal sensor 1301, the proximal sensor 1301 may send an indication of this interaction to the processing components 1430. The processing components 1430 may then send one or more signals to cause the first lighting state change to be performed. Processing components 1430 may be components that are programmed, or otherwise instructed, to perform the operations that they execute as described herein, and that may be included in one or more integrated circuit (IC) chips and/or other IC components.

In one specific example, the first lighting state change may include powering-on light source 1311, such as to cause light source 1311 to emit light. In FIG. 9, the proximal sensor 1301, distal sensor 1302 and processing components 1430 are powered by battery 1404. The light source 1311 may initially be unpowered, and the light source 1311 may remain unpowered until the movable component 1420 interacts with the proximal sensor 1301. When the movable component 1420 interacts with the proximal sensor 1301, the light source 1311 may be powered-on. Switch 1401 is an electrical switch that may control the flow of power from the battery 1404 to the light source 1311. The switch 1401 may be initially in an open state (which does not allow power to be provided to the light source 1311) and then, upon determining that the movable component 1420 interacts with the proximal sensor 1301, moved to a closed state (which does allow power to be provided to the light source 1311). In some examples, the processing components 1430 may, upon determining that the movable component 1420 interacts with the proximal sensor 1301, provide a signal to close the switch 1401 and allow power to flow to the light source 1311. Processing components 1430 may additionally or alternatively provide one or more other signals, for example to light source 1311, to perform other state changes, such as color changes and the like.

In some examples, the light source 1311 may also, after being powered-on, flash on, and off. The flashing may be a visual cue to continue the injection. In some examples, the processing components 1430 may, after determining that the movable component 1420 interacts with the proximal sensor 1301, provide signals to repeatedly open and close the switch 1101 to cause flashing of the light source 1311.

In some examples, a second lighting state change may be performed based on the distal sensor 1302 detecting interaction of the movable component 1420 with the distal sensor 1302. The second lighting state change may be a visual cue that the complete dosage of the medication has been delivered from the medication delivery device 1410. The second lighting state change may include changing a state of light source 1311 and/or changing a state of a second light source (not shown in FIG. 9). In one specific example, the second lighting state change may include powering-off the light source 1311, such as to cause the light source 1311 to stop flashing and/or stop emitting light. However, other state changes may also be performed. For example, the second lighting stage change may include causing light source 1311 to start emitting light, causing light source 1311 to stop emitting light, causing light source 1311 to start flashing, causing light source 1311 to stop flashing, causing light source 1311 to change color, and/or other state changes. The second lighting stage change may also include causing a second light source to start emitting light, causing the second light source to stop emitting light, causing the second light source to start flashing, causing the second light source to stop flashing, causing the second light source to change color, and/or other state changes. In some examples, upon detecting the interaction of the movable component 1420 with the distal sensor 1302, the distal sensor 1302 may send an indication of this interaction to the processing components 1430. The processing components 1430 may then send one or more signals to cause the second lighting state change to be performed. In some examples, the processing components 1430 may, after determining that the movable component 1420 interacts with the distal sensor 1302, provide signals to close the switch 1401 to cause power to stop being provided to the light source 1311.

In some examples, after determining that the movable component 1420 interacts with the distal sensor 1302, the light source 1311 may flash/pulse two or three times before being powered off. The second lighting state change may help to prevent the user from stopping the injection prematurely before the full dose is delivered. The second lighting state change may also cue to the user to stop pushing down on the medication delivery device 1410 and/or to lift up the medication delivery device 1410. In one specific example, the medication delivery device 1410 may include corresponding instructions, such as text that states, “stop pressing down when the light on the side turns off.” In some examples, audio may be provided via an attached speaker, such as that indicates to stop pressing when the light turns off (or another similar indication).

In some examples, any, or all, of the lighting circuit components (e.g., proximal sensor 1301, light source 1311, switch 1401, distal sensor 1302, processing components 1430, battery 1404) of the lighting circuit 1400 (as well as other lighting circuits described herein) may be implemented, in whole or in part, using one or more integrated circuit (IC) components, such as one or more IC chips. The integrated circuit components may be connected to one or more circuit boards, such as a printed circuit board and/or flexible circuit board. The components may communicate and/or interact with one another via one or more electrical connections, for example via the one or more circuit boards. Any, or all, of the lighting circuit components of the lighting circuit 1400 shown in FIG. 9 (as well as other lighting circuits described herein) may optionally include one or more processing components (e.g., integrated with or separate from processing components 1430) and/or one or more memory components. The memory components can be volatile (such as some types of RAM), non-volatile (such as ROM, flash memory, etc.), or a combination thereof.

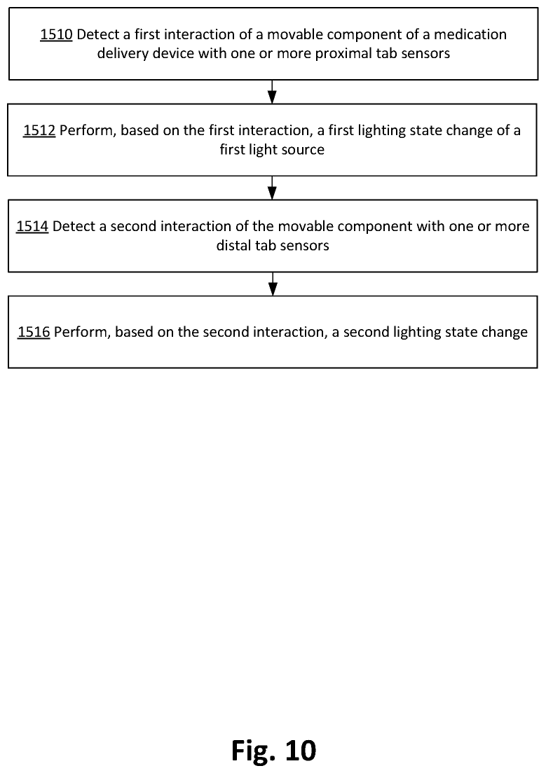

Referring now to FIG. 10, another light-based medication delivery cueing process will now be described in detail. The process of FIG. 10 is initiated at operation 1510, at which a first interaction is detected of a movable component of a medication delivery device with one or more proximal sensors, which are integrated with the medication delivery device. As described above, a movable component is a component of a medication delivery device that is moved, in an insertion direction (e.g., direction X₂ in FIG. 1A), to result in an injection of a medication into a patient. In the example of FIG. 8, upper housing 308 is a movable component of device 300. In the example of FIG. 9, proximal sensor 1301 detects when a movable component 1420 (e.g., upper housing 308) of the medication delivery device 1410 interacts with proximal sensor 1301. In some examples, there may be a plurality of proximal sensors. The plurality of proximal sensors may each be positioned at the same distance as one another from the distal end D, such that the upper housing simultaneously interacts with each of the plurality of proximal sensors. As described above, in some examples, the one or more proximal sensors may include a tactile sensor and/or a force sensor, and the first interaction may include contacting of the one or more proximal sensors by the movable component. Also, in some examples, the one or more proximal sensors may include a light sensor, and the first interaction may include a reduction in light detection due to covering of the light sensor by the movable component. Also, in some examples, the one or more proximal sensors may include a magnetic sensor, and the first interaction may include a movement of a metallic component of the movable component within a threshold distance of the magnetic sensor.

At operation 1512, a first lighting state change of a first light source is performed based on the first interaction. The first light source may be, for example, an OLED, OLED display, LED, or another type of light source. For example, when the proximal sensor 1301 detects that the movable component 1420 interacts with the proximal sensor 1301, the proximal sensor 1301 may send an indication of this first interaction to the processing components 1430. The processing components 1430 may then send one or more signals to cause the first lighting state change to be performed. In this manner, the first lighting state change may be performed based on the interaction of the movable component 1420 with the proximal sensor 1301. The first lighting state change may be a visual cue that the injection of the medication has begun. The first lighting stage change may include causing the first light source to start emitting light, causing the first light source to stop emitting light, causing the first light source to start flashing, causing the first light source to stop flashing, causing the first light source to change color, and/or other state changes. In one specific example, the first lighting state change may include powering-on the first light source, such as to cause the first light source to start emitting light. In the example of FIG. 6, the light source 1311 may initially be unpowered, and the light source 1311 may remain unpowered until the movable component 1420 interacts with the proximal sensor 1301. When the movable component 1420 interacts with the proximal sensor 1301, the light source 1311 may be powered-on. Switch 1401 is an electrical switch that may control the flow of power from the battery 1404 to the light source 1311. The switch 1401 may be initially in an open state (which does not allow power to be provided to the light source 1311) and then, upon determining that the movable component 1420 interacts with the proximal sensor 1301, moved to a closed state (which does allow power to be provided to the light source 1311). In some examples, the processing components 1430 may, upon determining that the movable component 1420 interacts with the proximal sensor 1301, provide a signal to close the switch 1401 and allow power to flow to the light source 1311. In some examples, the first light source may, after being powered on, flash on and off. The flashing may be a visual cue to continue the injection. In some examples, the processing components 1430 may, after determining that the movable component 1420 interacts with the proximal sensor 1301, provide signals to repeatedly open and close the switch 1101 to cause flashing of the light source 1311.

At operation 1514, a second interaction is detected of the movable component 1420 with one or more distal sensors, which are integrated with the medication delivery device. As described above, when being moved in the insertion direction to result in the injection of the medication into the patient, the movable component interacts with the one or more distal sensors after interacting with the one or more proximal sensors. In the example of FIG. 9, distal sensor 1302, detects when a movable component 1420 (e.g., upper housing 308) of the medication delivery device 1410 interacts with distal sensor 1302. In some examples, there may be a plurality of distal sensors. The plurality of distal sensors may each be positioned at the same distance as one another from the distal end D, such that the upper housing simultaneously interacts with each of the plurality of distal sensors. As described above, in some examples, the one or more distal sensors may include a tactile sensor and/or a force sensor, and the second interaction may include contacting of the one or more distal sensors by the movable component. Also, in some examples, the one or more distal sensors may include a light sensor, and the second interaction may include a reduction in light detection due to covering of the light sensor by the movable component. Also, in some examples, the one or more distal sensors may include a magnetic sensor, and the second interaction may include a movement of a metallic component of the movable component within a threshold distance of the magnetic sensor.

At operation 1516, a second lighting state change is performed based on the second interaction. The second lighting state change may be a visual cue that the complete dosage of the medication has been delivered from the medication delivery device. The second lighting state change may include changing a state of the first light source and/or changing a state of a second light source. The second light source may be, for example, an OLED, OLED display, LED, or another type of light source. In one specific example, the second lighting state change may include powering-off the first light source, such as to cause the first light source to stop flashing and/or stop emitting light. However, other state changes may also be performed. For example, the second lighting stage change may include causing the first light source to start emitting light, causing the first light source to stop emitting light, causing the first light source to start flashing, causing the first light source to stop flashing, causing the first light source to change color, and/or other state changes. The second lighting stage change may also include causing a second light source to start emitting light, causing the second light source to stop emitting light, causing the second light source to start flashing, causing the second light source to stop flashing, causing the second light source to change color, and/or other state changes. In some examples, upon detecting the interaction of the movable component 1420 with the distal sensor 1302, the distal sensor 1302 may send an indication of this interaction to the processing components 1430. The processing components 1430 may then send one or more signals to cause the second lighting state change to be performed. In some examples, the processing components 1430 may, after determining that the movable component 1420 interacts with the distal sensor 1302, provide signals to close the switch 1101 to cause power to stop being provided to the light source 1311.

While example embodiments of devices for executing the disclosed techniques are described herein, the underlying concepts can be applied to any system capable of performing the techniques described herein. Thus, the methods and apparatuses described herein can be implemented, or certain aspects or portions thereof, can take the form of program code (e.g., instructions) embodied in tangible non-transitory storage media (e.g., the one or more memory components described above), including a processor-readable or machine-readable storage medium, wherein, when the program code (e.g., instructions) is loaded into and executed by a machine, the machine becomes an apparatus for performing the techniques described herein.

While the techniques described herein can be implemented and have been described in connection with the various embodiments of the various figures, it is to be understood that other similar embodiments can be used or modifications and additions can be made to the described embodiments without deviating therefrom. For example, it should be appreciated that the steps disclosed above can be performed in the order set forth above, or in any other order as desired. The techniques described herein should not be limited to any single embodiment, but rather should be construed in breadth and scope in accordance with the appended claims.

1. A light-based medication delivery cueing system, comprising:
one or more force sensors, wherein the one or more force sensors measure an applied force that is applied, in an insertion direction, to a movable component of a medication delivery device, wherein the movable component is moved, in the insertion direction, to result in an injection of a medication into a patient, and wherein the one or more force sensors further measure a resistive force that resists movement, in the insertion direction, of the movable component;
one or more processing components, wherein the one or more processing components determine when the applied force becomes greater than, or equal to, a threshold break force that is required to initiate movement of the movable component in the insertion direction, and wherein the one or more processing components further determine when the resistive force becomes greater than, or equal to, a threshold back force that stops the movement of the movable component in the insertion direction;
a first light source, wherein a first lighting state change of the first light source is performed based on the applied force becoming greater than, or equal to, the threshold break force, and wherein a second lighting state change is performed based on the resistive force becoming greater than, or equal to, the threshold back force.

2. The light-based medication delivery cueing system of claim 1, wherein the one or more force sensors, the one or more processing components, and the first light source are included in one or more adhesive labels that are attached to the medication delivery device.

3. The light-based medication delivery cueing system of claim 1, wherein the one or more force sensors, the one or more processing components, and the first light source are included within the medication delivery device.

4. The light-based medication delivery cueing system of claim 1, wherein the one or more processing components further determine when the applied force is greater than, or equal to, a threshold glide force that is required to continue the movement of the movable component in the insertion direction.

5. The light-based medication delivery cueing system of claim 4, wherein a visual cue to continue the injection is provided based on the applied force being greater than, or equal to, the threshold glide force.

6. The light-based medication delivery cueing system of claim 5, wherein the visual cue comprises a flashing on, and off, of the first light source.

7. The light-based medication delivery cueing system of claim 1, wherein at least one of the one or more force sensors is contacted by a user during the injection.

8. The light-based medication delivery cueing system of claim 1, wherein at least one of the one or more force sensors is not contacted by a user during the injection.

9. The light-based medication delivery cueing system of claim 1, wherein the first light source is an organic light-emitting diode (OLED).

10. The light-based medication delivery cueing system of claim 1, wherein the first lighting state change comprises at least one of causing the first light source to start emitting light, changing a color of the first light source, or causing the first light source to start flashing.

11. The light-based medication delivery cueing system of claim 1, wherein the second lighting state change comprises at least one of causing the first light source to stop emitting light, changing a color of the first light source, or causing the first light source to stop flashing.

12. The light-based medication delivery cueing system of claim 1, further comprising a second light source.

13. The light-based medication delivery cueing system of claim 12, wherein the second lighting state change comprises at least one of causing the second light source to start emitting light, changing a color of the second light source, or causing the second light source to start flashing.

14. The light-based medication delivery cueing system of claim 12, wherein the first light source has a different color than the second light source.

15. The light-based medication delivery cueing system of claim 1, wherein the second lighting state change is a visual cue that a complete dosage of the medication has been delivered from the medication delivery device.

16. A light-based medication delivery cueing method comprising:
measuring, by one or more force sensors, an applied force that is applied, in an insertion direction, to a movable component of a medication delivery device, wherein the movable component is moved, in the insertion direction, to result in an injection of a medication into a patient;
measuring, by the one or more force sensors, a resistive force that resists movement, in the insertion direction, of the movable component;
determining when the applied force becomes greater than, or equal to, a threshold break force that is required to initiate movement of the movable component in the insertion direction;
performing a first lighting state change of a first light source based on the applied force becoming greater than, or equal to, the threshold break force;
determining when the resistive force becomes greater than, or equal to, a threshold back force that stops the movement of the movable component in the insertion direction; and
performing a second lighting state change based on the resistive force becoming greater than, or equal to, the threshold back force.

17-29. (canceled)

30. A light-based medication delivery cueing system, comprising:
one or more proximal sensors that detect a first interaction of a movable component of a medication delivery device with the one or more proximal sensors, wherein the movable component is moved, in an insertion direction, to result in an injection of a medication into a patient,
one or more distal sensors that detect a second interaction of the movable component with the one or more distal sensors, wherein, when being moved in the insertion direction to result in the injection, the movable component interacts with the one or more distal sensors after interacting with the one or more proximal sensors; and
a first light source, wherein a first lighting state change of the first light source is performed based on the first interaction, and wherein a second lighting state change is performed based on the second interaction.

31-32. (canceled)

33. The light-based medication delivery cueing system of claim 30, wherein the one or more proximal sensors comprise at least one of a tactile sensor or a force sensor, and wherein the first interaction comprises contacting of the one or more proximal sensors by the movable component.

34. The light-based medication delivery cueing system of claim 30, wherein the one or more distal sensors comprise at least one of a tactile sensor or a force sensor, and wherein the second interaction comprises contacting of the one or more distal sensors by the movable component.

35. The light-based medication delivery cueing system of claim 30, wherein the one or more proximal sensors comprise a light sensor, and wherein the first interaction comprises a reduction in light detection due to covering of the light sensor by the movable component.

36-58. (canceled)
